(19) United States
(12) Patent Application Publication
(10) Pub. No.: US 2022/0117942 A1
(43) Pub. Date: Apr. 21, 2022

(54) COMBINATION THERAPY FOR THE TREATMENT OF PROSTATE CANCER

(71) Applicant: Zenith Epigenetics Ltd., Calgary (CA)

(72) Inventors: Sarah Christine ATTWELL, Calgary (CA); Eric CAMPEAU, Calgary (CA); Sanjay LAKHOTIA, Orinda, CA (US)

(73) Assignee: Zenith Epigenetics Ltd., Calgary, AB (CA)

(21) Appl. No.: 17/275,473

(22) PCT Filed: Sep. 13, 2019
(86) PCT No.: PCT/US2019/050970

§ 371 Date: Mar. 11, 2021

Related U.S. Application Data
(60) Provisional application No. 62/730,869, filed on Sep. 13, 2018.
(60) Provisional application No. 62/737,612, filed on Sep. 27, 2018.
(60) Provisional application No. 62/778,185, filed on Dec. 11, 2018.

(51) Int. Cl. A61K 31/4166; A61K 31/58; A61P 35/00; A61K 45/06

(52) U.S. Cl. CPC A61K 31/4166; A61K 45/06; A61P 35/00; A61K 31/58

(57) ABSTRACT

The invention provides methods for treating prostate cancer, including metastatic castration-resistant prostate cancer, comprising administering to a subject in need thereof a BET bromodomain inhibitor in combination with a second agent.

This application is a U.S. national phase entry under 35 U.S.C. § 371 from International Application No. PCT/US2019/050970, filed Sep. 13, 2019, which claims the benefit of priority of U.S. Provisional Application No. 62/730,869, filed Sep. 13, 2018, U.S. Provisional Application No. 62/737,612, filed Sep. 27, 2018, and U.S. Provisional Application No. 62/778,185, filed Dec. 11, 2018, all of which are incorporated herein by reference in their entirety.

The invention relates to a combination therapy for the treatment of prostate cancer.

### BACKGROUND

Metastatic castration-resistant prostate cancer (“mCRPC”) is often characterized by the persistence of signaling of the androgen receptor (“AR”) to drive cancer proliferation, tumor invasion, and metastasis (Wyatt & Gleave, 2015). Initial therapies of prostate cancer include either surgical or chemical castration, followed by androgen-deprivation therapy. In many instances, further progression and metastases of the cancer is observed, hence the term metastatic castration resistant prostate cancer. First line standard of care therapies for mCRPC include the AR antagonist enzalutamide, androgen synthesis inhibitors such as the cytochrome steroid 17-alpha-hydroxylase/17,20 lyase (CYP17A1) inhibitor abiraterone and in some cases chemotherapy. However, recent studies have shown that subjects become resistant to these first-line treatments over time and require additional drug therapy (Wyatt & Gleave, 2015). Currently, there is no standard of care for second-line mCRPC as the efficacy of AR modulators or chemotherapy in the second-line setting is moderate. Furthermore, it has been suggested that the resistance mechanisms of abiraterone and enzalutamide overlap (Azad et al, 2015b; Bianchini et al, 2014; Loriot et al, 2013; Noonan et al, 2013; Schrader et al, 2014).

Mechanisms of resistance to enzalutamide and abiraterone include alternative splicing of the AR resulting in the loss of the ligand binding domain and constitutively active androgen signaling (Nakazawa et al, 2014), up-regulation of alternate pathways such as glucocorticoid receptor (GR) (Arora et al, 2013; Isikbay et al, 2014), nuclear factor kappa-light-chain-enhancer of activated B cells (NF-κB) (Jin et al, 2013; Nadiminty et al, 2013), or MYC signaling pathways (Lamb et al, 2014; Nadiminty et al, 2013; Zeng et al, 2015), as well as neuroendocrine differentiation (Aggarwal et al, 2014; Beltran et al, 2014; Dang et al, 2015). Several of these resistance mechanisms have either been shown to be regulated by the BET proteins in prostate cancer (MYC expression: (Gao et al, 2013); AR splice variants: (Chan et al, 2015; Welti et al, 2018); GR: (Asangani et al, 2016; Shah et al, 2017)) or in other cancers (NF-κB: (Ceribelli et al, 2014; Gallagher et al, 2014; Zou et al, 2014)), suggesting that BET inhibition could be beneficial for subjects with mCRPC that are resistant to enzalutamide and abiraterone. In particular, the androgen receptor splice variant 7 (AR-V7) was recently suggested to be involved in the resistance to enzalutamide and abiraterone (Antonarakis et al, 2014); cell lines expressing these variants are BET-dependent and sensitive to BETi in culture and in xenografts (Asangani et al, 2014; Asangani et al, 2016; Chan et al, 2015; Gao et al, 2013; Wyce et al, 2013). One of the proposed mechanisms of action of the BET inhibitor (BETi) is to prevent the BET proteins from interacting with the N-terminus of the AR and activating downstream androgen signaling pathways (Asangani et al, 2014).

However, at this time, it is unclear which, if any, BET inhibitors will result in significant clinical benefit when administered to subjects with prostate cancer, particularly mCRPC. It is also unclear which, if any BET inhibitors will combine synergistically with other drugs, such as an androgen receptor antagonist or an androgen synthesis inhibitor, in the treatment of prostate cancer; what level of synergy is required; and which second therapeutic agent will be the best combination partner for each BET inhibitor, resulting in clinical benefit when administered to patients with prostate cancer. In addition to a clinical benefit, the combination also has to be safe and well tolerated at the efficacious doses. At this time, it cannot be predicted which combination will show the best overall profile.

### SUMMARY

The present invention provides methods of treating prostate cancer by co-administration of a BET bromodomain inhibitor, or a pharmaceutically acceptable salt or co-crystal of a BET bromodomain inhibitor, and a second therapeutic agent to a subject in need thereof.

In some embodiments, the BET bromodomain inhibitor is administered simultaneously with the second therapeutic agent. In some embodiments, the BET bromodomain inhibitor is administered sequentially with the second therapeutic agent. In some embodiments, the BET bromodomain inhibitor is administered in a single pharmaceutical composition with the second therapeutic agent. In some embodiments, the BET bromodomain inhibitor and the second therapeutic agent are administered as separate compositions.

In some embodiments the second therapeutic agent is an agent beneficial to the treatment of prostate cancer.

In some embodiments, the second is therapeutic agent is an androgen-deprivation therapy. In some embodiments, the second therapeutic is an androgen receptor antagonist. In some embodiments, the second therapeutic is an androgen synthesis inhibitor.

In some embodiments, the prostate cancer is metastatic castration-resistant prostate cancer (mCRPC).

In some embodiments, the BET bromodomain inhibitor is a compound of Formula Ia or Formula Ib

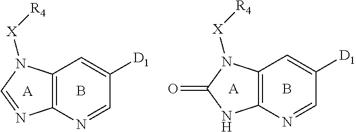

or a stereoisomer, tautomer, pharmaceutically acceptable salt, or co-crystal, or hydrate thereof, wherein:

Ring A and Ring B may be optionally substituted with groups independently selected from hydrogen, deuterium, —NH₂, amino, heterocycle(C₄-C₆), carbocycle(C₄-C₆), halogen, —CN, —OH, —CF₃, alkyl (C₁-C₆), thioalkyl (C₁-C₆), alkenyl (C₂-C₆), and alkoxy (C₁-C₆); X is selected from —NH—, —CH₂—, —CH₂CH₂—, —CH₂CH₂CH₂—, —CH₂CH₂O—, —CH₂CH₂NH—, —CH₂CH₂S—, —C(O)—, —C(O)CH₂—, —C(O)CH₂CH₂—, —CH₂C(O)—, —CH₂CH₂C(O)—, —C(O)NH—, —C(O)O—, —C(O)S—, —C(O)NHCH₂—, —C(O)OCH₂—, —C(O)SCH₂—, wherein one or more hydrogen may independently be replaced with deuterium, hydroxyl, methyl, halogen, —CF₃, ketone, and where S may be oxidized to sulfoxide or sulfone;

R₄ is selected from optionally substituted 3-7 membered carbocycles and heterocycles; and

D₁ is selected from the following 5-membered monocyclic heterocycles:

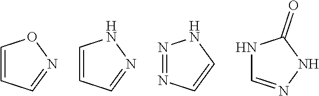

which are optionally substituted with hydrogen, deuterium, alkyl (C₁-C₄), alkoxy (C₁-C₄), amino, halogen, amide, —CF₃, —CN, —N₃, ketone (C₁-C₄), —S(O)Alkyl(C₁-C₄), —SO₂alkyl(C₁-C₄), -thioalkyl(C₁-C₄), —COOH, and/or ester, each of which may be optionally substituted with hydrogen, F, Cl, Br, —OH, —NH₂, —NHMe, —OMe, —SMe, oxo, and/or thio-oxo.

In some embodiments, the BET bromodomain inhibitor is 1-benzyl-6-(3,5-dimethylisoxazol-4-yl)-N-methyl-1H-imidazo[4,5-b]pyridine-2-amine, herein Compound I, has the following formula:

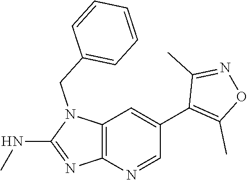 [SMILES: CNc1nc2ncc(-c3c(C)noc3C)cc2n1Cc1ccccc1]

In some embodiments, the BET bromodomain inhibitor is Compound I or a pharmaceutically acceptable salt or co-crystal. In some embodiments, the BET bromodomain inhibitor is a mesylate salt/co-crystal of Compound I in crystalline form I.

In some embodiments, the combination therapy of the invention demonstrates an unexpected superior safety profile because it does not result in dose limiting toxicity due to thrombocytopenia. In some embodiments, the combination therapy of the invention demonstrates synergistic therapeutic effects.

### BRIEF DESCRIPTION OF THE DRAWINGS

FIG. 1 shows the effect (inhibition) of Compound I, enzalutamide, and the combination of Compound I and enzalutamide on cell proliferation of VCaP cells (AR-positive, AR amplified, TMPRSS2-ERG fusion).

FIG. 2 shows the effect (inhibition) of Compound I, apalutamide (ARN-509), and the combination of Compound I and apalutamide on cell proliferation of VCaP cells (AR-positive, AR amplified, TMPRSS2-ERG fusion).

FIG. 3 shows the effect (inhibition) of Compound I, abiraterone, and the combination of Compound I and abiraterone on proliferation of LAPC4 cells.

FIG. 4 shows an X-ray powder diffractogram (XRPD) of a mesylate salt/co-crystal of Compound I.

FIG. 5 shows a differential scanning calorimeter (DSC) curve of a mesylate salt/co-crystal of Compound I.

FIG. 6 shows a thermogravimetric analysis (TGA) of a mesylate salt/co-crystal of Compound I.

FIG. 7 shows the Kaplan-Meier survival curves of patients treated with Compound I and enzalutamide that previously progressed on either abiraterone or enzalutamide and all patients. The number of patients, events and median progression-free survival (PFS) are depicted in the table below.

FIG. 8 shows the Kaplan-Meier curves of patients treated with Compound I and enzalutamide that had either a PSA response, PSA spike, or neither (no PSA modulation) after 12 weeks of treatment. The number of patients, events, and median progression-free survival (PFS) are depicted in the table below.

FIG. 9 shows an example of four mCRPC patients treated QD with Compound I in combination with enzalutamide that have a PSA spike at either week 4 or week 8

FIG. 10 shows the distribution of ETS mutations or fusions in mCRPC patients treated QD with Compound I in combination with enzalutamide and whether they responded (>24 weeks without clinical or radiographic progression) or did not respond (≤24 weeks before radiographic or clinical progression).

FIG. 11 shows the distribution of ETS mutations or fusions in mCRPC patients treated QD with Compound I in combination with enzalutamide and whether they had a PSA spike or PSA response at either week 4 or week 8. Responders are defined by >24 weeks post dosing with Compound I without clinical or radiographic progression and Non-Responders by ≤24 weeks before radiographic or clinical progression.

FIG. 12A shows the induction of the immune response in the tumor in response to the combination of Compound I with enzalutamide in mCRPC patients. Enzalutamide was continually present in both the pre-Compound I and post-Compound I sample. FIG. 12B shows some of the immune response genes that were upregulated in the tumor.

### DEFINITIONS

As used herein, “treatment” or “treating” refers to an amelioration of a disease or disorder, or at least one discernible symptom thereof. In another embodiment, “treatment” or “treating” refers to an amelioration of at least one measurable physical parameter, not necessarily discernible by the subject. In yet another embodiment, “treatment” or “treating” refers to inhibiting the progression of a disease or disorder, either physically, e.g., stabilization of a discernible symptom, physiologically, e.g., stabilization of a physical parameter, or both. In yet another embodiment, “treatment” or “treating” refers to delaying the onset of a disease or disorder.

By “optional” or “optionally” is meant that the subsequently described event or circumstance may or may not occur, and that the description includes instances where the event or circumstance occurs and instances in which is does not. For example, “optionally substituted aryl” encompasses both “aryl” and “substituted aryl” as defined below. It will be understood by those skilled in the art, with respect to any group containing one or more substituents, that such groups are not intended to introduce any substitution or substitution patterns that are sterically impractical, synthetically non-feasible and/or inherently unstable.

As used herein, the term “hydrate” refers to a crystal form with either a stoichiometric or non-stoichiometric amount of water is incorporated into the crystal structure.

The term “alkenyl” as used herein refers to an unsaturated straight or branched hydrocarbon having at least one carbon-carbon double bond, such as a straight or branched group of 2-8 carbon atoms, referred to herein as (C₂-C₈) alkenyl. Exemplary alkenyl groups include, but are not limited to, vinyl, allyl, butenyl, pentenyl, hexenyl, butadienyl, pentadienyl, hexadienyl, 2-ethylhexenyl, 2-propyl-2-butenyl, and 4-(2-methyl-3-butene)-pentenyl.

The term “alkoxy” as used herein refers to an alkyl group attached to an oxygen (—O— alkyl-). “Alkoxy” groups also include an alkenyl group attached to an oxygen (“alkenyloxy”) or an alkynyl group attached to an oxygen (“alkynyloxy”) groups. Exemplary alkoxy groups include, but are not limited to, groups with an alkyl, alkenyl or alkynyl group of 1-8 carbon atoms, referred to herein as (C₁-C₈) alkoxy. Exemplary alkoxy groups include, but are not limited to, methoxy and ethoxy.

The term “alkyl” as used herein refers to a saturated straight or branched hydrocarbon, such as a straight or branched group of 1-8 carbon atoms, referred to herein as (C₁-C₈) alkyl. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, and octyl.

The term “amide” as used herein refers to —NRₐC(O)(R_{b}), or —C(O)NR_{b}R_{c}, wherein Rₐ, R_{b} and R_{c} are each independently selected from alkyl, alkenyl, alkynyl, aryl, arylalkyl, cycloalkyl, haloalkyl, heteroaryl, heterocyclyl, and hydrogen. The amide can be attached to another group through the carbon, the nitrogen, Rₐ, R_{b}, or R_{c}. The amide also may be cyclic, for example R_{b} and R_{c}, may be joined to form a 3- to 8-membered ring, such as 5- or 6-membered ring. The term “amide” encompasses groups such as sulfonamide, urea, ureido, carbamate, carbamic acid, and cyclic versions thereof. The term “amide” also encompasses an amide group attached to a carboxy group, e.g., -amide-COOH or salts such as -amide-COONa, an amino group attached to a carboxy group (e.g., -amino-COOH or salts such as -amino-COONa).

The term “amine” or “amino” as used herein refers to the form —NR_{d}Rₑ or —N(R_{d})Rₑ—, where R_{d} and Rₑ are independently selected from alkyl, alkenyl, alkynyl, aryl, arylalkyl, carbamate, cycloalkyl, haloalkyl, heteroaryl, heterocycle, and hydrogen. The amino can be attached to the parent molecular group through the nitrogen. The amino also may be cyclic, for example any two of R_{d} and Rₑ may be joined together or with the N to form a 3- to 12-membered ring (e.g., morpholino or piperidinyl). The term amino also includes the corresponding quaternary ammonium salt of any amino group. Exemplary amino groups include alkylamino groups, wherein at least one of R_{d} or Rₑ is an alkyl group. In some embodiments R_{d} and Rₑ each may be optionally substituted with hydroxyl, halogen, alkoxy, ester, or amino.

The term “aryl” as used herein refers to a mono-, bi-, or other multi-carbocyclic, aromatic ring system. The aryl group can optionally be fused to one or more rings selected from aryls, cycloalkyls, and heterocyclyls. The aryl groups of this present disclosure can be substituted with groups selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Exemplary aryl groups also include, but are not limited to, a monocyclic aromatic ring system, wherein the ring comprises 6 carbon atoms, referred to herein as “(C₆) aryl.”

The term “arylalkyl” as used herein refers to an alkyl group having at least one aryl substituent (e.g., -aryl-alkyl-). Exemplary arylalkyl groups include, but are not limited to, arylalkyls having a monocyclic aromatic ring system, wherein the ring comprises 6 carbon atoms, referred to herein as “(C₆) arylalkyl.”

The term “carbamate” as used herein refers to the form —R_{g}OC(O)N(Rₕ)—, —R_{g}OC(O)N(Rₕ)Rᵢ—, or —OC(O)NRₕRᵢ, wherein R_{g}, Rₕ and Rᵢ are each independently selected from alkyl, alkenyl, alkynyl, aryl, arylalkyl, cycloalkyl, haloalkyl, heteroaryl, heterocyclyl, and hydrogen. Exemplary carbamates include, but are not limited to, arylcarbamates or heteroaryl carbamates (e.g., wherein at least one of R_{g}, Rₕ and Rᵢ are independently selected from aryl or heteroaryl, such as pyridine, pyridazine, pyrimidine, and pyrazine).

The term “carbocycle” as used herein refers to an aryl or cycloalkyl group.

The term “carboxy” as used herein refers to —COOH or its corresponding carboxylate salts (e.g., —COONa). The term carboxy also includes “carboxycarbonyl,” e.g. a carboxy group attached to a carbonyl group, e.g., —C(O)—COOH or salts, such as —C(O)—COONa.

The term “cycloalkoxy” as used herein refers to a cycloalkyl group attached to an oxygen.

The term “cycloalkyl” as used herein refers to a saturated or unsaturated cyclic, bicyclic, or bridged bicyclic hydrocarbon group of 3-12 carbons, or 3-8 carbons, referred to herein as “(C₃-C₈) cycloalkyl,” derived from a cycloalkane. Exemplary cycloalkyl groups include, but are not limited to, cyclohexanes, cyclohexenes, cyclopentanes, and cyclopentenes. Cycloalkyl groups may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Cycloalkyl groups can be fused to other cycloalkyl saturated or unsaturated, aryl, or heterocyclyl groups.

The term “dicarboxylic acid” as used herein refers to a group containing at least two carboxylic acid groups such as saturated and unsaturated hydrocarbon dicarboxylic acids and salts thereof. Exemplary dicarboxylic acids include alkyl dicarboxylic acids. Dicarboxylic acids may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydrogen, hydroxyl, ketone, nitro, phosphate, sulfide, sulfinyl, sulfonyl, sulfonic acid, sulfonamide and thioketone. Dicarboxylic acids include, but are not limited to succinic acid, glutaric acid, adipic acid, suberic acid, sebacic acid, azelaic acid, maleic acid, phthalic acid, aspartic acid, glutamic acid, malonic acid, fumaric acid, (+)/(−)-malic acid, (+)/(−) tartaric acid, isophthalic acid, and terephthalic acid. Dicarboxylic acids further include carboxylic acid derivatives thereof, such as anhydrides, imides, hydrazides (for example, succinic anhydride and succinimide).

The term “ester” refers to the structure —C(O)O—, —C(O)O—Rⱼ—, —RₖC(O)O—Rⱼ—, or —RₖC(O)O—, where O is not bound to hydrogen, and Rⱼ and Rₖ can independently be selected from alkoxy, aryloxy, alkyl, alkenyl, alkynyl, amide, amino, aryl, arylalkyl, cycloalkyl, ether, haloalkyl, heteroaryl, and heterocyclyl. Rₖ can be a hydrogen, but Rⱼ cannot be hydrogen. The ester may be cyclic, for example the carbon atom and Rⱼ, the oxygen atom and Rₖ, or Rⱼ and Rₖ may be joined to form a 3- to 12-membered ring. Exemplary esters include, but are not limited to, alkyl esters wherein at least one of Rⱼ or Rₖ is alkyl, such as —O—C(O)-alkyl, —C(O)—O-alkyl-, and -alkyl-C(O)—O-alkyl-. Exemplary esters also include aryl or heteoraryl esters, e.g. wherein at least one of Rⱼ or Rₖ is a heteroaryl group such as pyridine, pyridazine, pyrimidine and pyrazine, such as a nicotinate ester. Exemplary esters also include reverse esters having the structure —RₖC(O)O—, where the oxygen is bound to the parent molecule. Exemplary reverse esters include succinate, D-argininate, L-argininate, L-lysinate and D-lysinate. Esters also include carboxylic acid anhydrides and acid halides.

The terms “halo” or “halogen” as used herein refer to F, Cl, Br, or I.

The term “haloalkyl” as used herein refers to an alkyl group substituted with one or more halogen atoms. “Haloalkyls” also encompass alkenyl or alkynyl groups substituted with one or more halogen atoms.

The term “heteroaryl” as used herein refers to a mono-, bi-, or multi-cyclic, aromatic ring system containing one or more heteroatoms, for example 1-3 heteroatoms, such as nitrogen, oxygen, and sulfur. Heteroaryls can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heteroaryls can also be fused to non-aromatic rings. Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3)- and (1,2,4)-triazolyl, pyrazinyl, pyrimidilyl, tetrazolyl, furyl, thienyl, isoxazolyl, thiazolyl, furyl, phenyl, isoxazolyl, and oxazolyl. Exemplary heteroaryl groups include, but are not limited to, a monocyclic aromatic ring, wherein the ring comprises 2-5 carbon atoms and 1-3 heteroatoms, referred to herein as “(C₂-C₅) heteroaryl.”

The terms “heterocycle,” “heterocyclyl,” or “heterocyclic” as used herein refer to a saturated or unsaturated 3-, 4-, 5-, 6- or 7-membered ring containing one, two, or three heteroatoms independently selected from nitrogen, oxygen, and sulfur. Heterocycles can be aromatic (heteroaryls) or non-aromatic. Heterocycles can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heterocycles also include bicyclic, tricyclic, and tetracyclic groups in which any of the above heterocyclic rings is fused to one or two rings independently selected from aryls, cycloalkyls, and heterocycles. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl.

The terms “hydroxy” and “hydroxyl” as used herein refer to —OH.

The term “hydroxyalkyl” as used herein refers to a hydroxy attached to an alkyl group.

The term “hydroxyaryl” as used herein refers to a hydroxy attached to an aryl group.

The term “ketone” as used herein refers to the structure —C(O)—Rₙ (such as acetyl, —C(O)CH₃) or —Rₙ—C(O)—Rₒ—. The ketone can be attached to another group through Rₙ or Rₒ. Rₙ or Rₒ can be alkyl, alkenyl, alkynyl, cycloalkyl, heterocyclyl or aryl, or Rₙ or Rₒ can be joined to form a 3- to 12-membered ring.

The term “phenyl” as used herein refers to a 6-membered carbocyclic aromatic ring. The phenyl group can also be fused to a cyclohexane or cyclopentane ring. Phenyl can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, phosphate, sulfide, sulfinyl, sulfonyl, sulfonic acid, sulfonamide and thioketone.

The term “thioalkyl” as used herein refers to an alkyl group attached to a sulfur (—S— alkyl-).

“Alkyl,” “alkenyl,” “alkynyl”, “alkoxy”, “amino” and “amide” groups can be optionally substituted with or interrupted by or branched with at least one group selected from alkoxy, aryloxy, alkyl, alkenyl, alkynyl, amide, amino, aryl, arylalkyl, carbamate, carbonyl, carboxy, cyano, cycloalkyl, ester, ether, formyl, halogen, haloalkyl, heteroaryl, heterocyclyl, hydroxyl, ketone, phosphate, sulfide, sulfinyl, sulfonyl, sulfonic acid, sulfonamide, thioketone, ureido and N. The substituents may be branched to form a substituted or unsubstituted heterocycle or cycloalkyl.

As used herein, a suitable substitution on an optionally substituted substituent refers to a group that does not nullify the synthetic or pharmaceutical utility of the compounds of the present disclosure or the intermediates useful for preparing them. Examples of suitable substitutions include, but are not limited to: C₁₋₈ alkyl, alkenyl or alkynyl; C₁₋₆ aryl, C₂₋₅ heteroaryl; C₃₇ cycloalkyl; C₁₋₈ alkoxy; C₆ aryloxy; —CN; —OH; oxo; halo, carboxy; amino, such as —NH(C₁₋₈ alkyl), —N(C₁₋₈ alkyl)₂, —NH((C₆)aryl), or —N((C₆)aryl)₂; formyl; ketones, such as —CO(C₁₋₈ alkyl), —CO((C₆aryl) esters, such as —CO₂(C₁₋₈ alkyl) and —CO₂ (C₆ aryl). One of skill in art can readily choose a suitable substitution based on the stability and pharmacological and synthetic activity of the compound of the present disclosure.

The term “pharmaceutically acceptable composition” as used herein refers to a composition comprising at least one compound as disclosed herein formulated together with one or more pharmaceutically acceptable carriers.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “disease progression” as used herein refers to an increase in prostate specific antigen (“PSA”) and/or progressing metastatic disease. In some embodiments, disease progression is defined as described in the Prostrate Cancer Working Group (PCWG)2 guidelines (Scher et al. 2008). In some embodiments, disease progression occurs in subjects who have previously received androgen deprivation therapy.

### Exemplary Embodiments of the Invention

As summarized above, the present invention provides methods of treating prostate cancer by concomitant administration of a BET bromodomain inhibitor, or a pharmaceutically acceptable salt or co-crystal of a BET bromodomain inhibitor, and a second therapeutic agent to a subject in need thereof.

In one embodiment, the invention provides a method for treating prostate cancer comprising concomitantly administrating to a subject in need thereof a BET bromodomain inhibitor of Formula Ia or Formula Ib

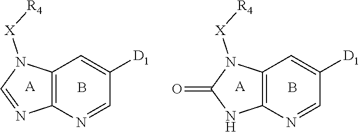

or a stereoisomer, tautomer, pharmaceutically acceptable salt, or co-crystal, or hydrate thereof, and a second therapeutic agent, wherein:

Ring A and Ring B may be optionally substituted with groups independently selected from hydrogen, deuterium, —NH₂, amino, heterocycle(C₄-C₆), carbocycle(C₄-C₆), halogen, —CN, —OH, —CF₃, alkyl (C₁-C₆), thioalkyl (C₁-C₆), alkenyl (C₁-C₆), and alkoxy (C₁-C₆);

X is selected from —NH—, —CH₂—, —CH₂CH₂—, —CH₂CH₂CH₂—, —CH₂CH₂O—, —CH₂CH₂NH—, —CH₂CH₂S—, —C(O)—, —C(O)CH₂—, —C(O)CH₂CH₂—, —CH₂C(O)—, —CH₂CH₂C(O)—, —C(O)NH—, —C(O)O—, —C(O)S—, —C(O)NHCH₂—, —C(O)OCH₂—, —C(O)SCH₂—, wherein one or more hydrogen may independently be replaced with deuterium, hydroxyl, methyl, halogen, —CF₃, ketone, and where S may be oxidized to sulfoxide or sulfone;

R₄ is selected from optionally substituted 3-7 membered carbocycles and heterocycles; and

D₁ is selected from the following 5-membered monocyclic heterocycles:

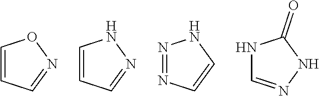

which are optionally substituted with hydrogen, deuterium, alkyl (C₁-C₄), alkoxy (C₁-C₄), amino, halogen, amide, —CF₃, —CN, —N₃, ketone (C₁-C₄), —S(O)Alkyl(C₁-C₄), —SO₂alkyl(C₁-C₄), -thioalkyl(C₁-C₄), —COOH, and/or ester, each of which may be optionally substituted with hydrogen, F, Cl, Br, —OH, —NH₂, —NHMe, —OMe, —SMe, oxo, and/or thio-oxo.

Compounds of Formula Ia and Ib, including Compound I, have been previously described in International Patent Publication WO 2015/002754, incorporated herein by reference in its entirety, and particularly for its description of the compounds of Formula Ia and Formula Ib, including Compound I, their synthesis, and the demonstration of their BET bromodomain inhibitor activity.

In some embodiments, the BET bromodomain inhibitor of Formula Ia or Formula Ib is selected from:
1-Benzyl-6-(3,5-dimethylisoxazol-4-yl)-N-ethyl-1H-imidazo[4,5-b]pyridin-2-amine;
1-Benzyl-6-(3,5-dimethylisoxazol-4-yl)-N-methyl-1H-imidazo[4,5-b]pyridin-2-amine;
N,1-Dibenzyl-6-(3,5-dimethylisoxazol-4-yl)-1H-imidazo[4,5-b]pyridin-2-amine;
1-Benzyl-6-(3,5-dimethylisoxazol-4-yl)-N-(pyridin-3-ylmethyl)-1H-imidazo[4,5-b]pyridin-2-amine;
4-(1-Benzyl-2-(pyrrolidin-1-yl)-1H-imidazo[4,5-b]pyridin-6-yl)-3,5-dimethylisoxazole;
4-(2-(Azetidin-1-yl)-1-(cyclopentylmethyl)-1H-imidazo[4,5-b]pyridin-6-yl)-3,5-dimethylisoxazole;
1-Benzyl-6-(3,5-dimethylisoxazol-4-yl)-1H-imidazo[4,5-b]pyridin-2-amine;
1-(cyclopentylmethyl)-6-(3,5-dimethylisoxazol-4-yl)-N-(tetrahydro-2H-pyran-4-yl)-1H-imidazo[4,5-b]pyridin-2-amine;
4-Amino-1-benzyl-6-(3,5-dimethylisoxazol-4-yl)-1H-benzo[d]imidazol-2(3H)-one;
4-Amino-6-(3,5-dimethylisoxazol-4-yl)-1-(4-methoxybenzyl)-1H-benzo[d]imidazol-2(3H)-one;
4-Amino-6-(3,5-dimethylisoxazol-4-yl)-1-(1-phenylethyl)-1H-benzo[d]imidazol-2(3H)-one;
4-Amino-1-benzyl-6-(3,5-dimethylisoxazol-4-yl)-3-methyl-1H-benzo[d]imidazol-2(3H)-one;
   or a pharmaceutically acceptable salt or co-crystal thereof.

In some embodiments, the invention provides a method for treating prostate cancer comprising administrating to a subject in need thereof, a compound selected from 1-benzyl-6-(3,5-dimethylisoxazol-4-yl)-N-methyl-1H-imidazo[4,5-b]pyridin-2-amine (Compound I) and 1-benzyl-6-(3,5-dimethylisoxazol-4-yl)-1H-imidazo[4,5-b]pyridin-2-amine and pharmaceutically acceptable salts or co-crystals thereof, concomitantly with another therapeutic agent.

In some embodiments, the invention provides a method for treating prostate cancer comprising administrating to a subject in need thereof, a compound selected from 1-benzyl-6-(3,5-dimethylisoxazol-4-yl)-N-methyl-1H-imidazo[4,5-b]pyridin-2-amine (Compound I) and 1-benzyl-6-(3,5-dimethylisoxazol-4-yl)-1H-imidazo[4,5-b]pyridin-2-amine and pharmaceutically acceptable salts or co-crystals thereof, concomitantly with both another therapeutic agent and an immune checkpoint inhibitor.

In one embodiment, the second agent is an androgen receptor antagonist.

In one embodiment, the second agent is an androgen synthesis inhibitor.

In one embodiment, the second agent is enzalutamide.

In one embodiment, the second agent is apalutamide.

In one embodiment, the second agent is darolutamide.

In one embodiment, the second agent is abiraterone.

In one embodiment, the second agent is an androgen receptor antagonist and is administered in combination with an immune checkpoint inhibitor.

In one embodiment, the second agent is an androgen synthesis inhibitor and is administered in combination with an immune checkpoint inhibitor

In some embodiments, the immune checkpoint inhibitor is a PD-1, PD-L1 inhibitor, or CTL-4 inhibitor.

In some embodiments, the immune checkpoint inhibitor is Ipilimumab, Nivolumab, Pembrolizumab PD-1, Atezolizumab, Avelumab, Durvalumab, or Cemiplimab.

In one embodiment, the prostate cancer is castration-resistant prostate cancer or metastatic castration-resistant prostate cancer.

In one embodiment, the subject previously has been treated with a prostate cancer therapy.

In one embodiment, the prostate cancer therapy is an androgen-deprivation therapy.

In one embodiment, the subject previously has shown disease progression on androgen-deprivation therapy.

In one embodiment, the patient is still responding to androgen deprivation therapy.

In one embodiment, the subject has not previously been treated with androgen-deprivation therapy.

In one embodiment, the androgen-deprivation therapy is enzalutamide, apalutamide, or abiraterone.

In one embodiment, the pharmaceutically acceptable salt or co-crystal is the mesylate salt or co-crystal.

In one embodiment, the subject has asymptomatic non-metastatic disease with rising PSA and negative scans for measurable disease.

In one embodiment, the subject has metastatic disease with rising PSA and positive scans for metastatic disease and has not been treated with androgen-deprivation therapy or chemotherapy (pre-taxane).

In one embodiment, the subject has metastatic disease with rising PSA and positive scans for metastatic disease, and has not been treated with abiraterone, enzalutamide, or apalutamide, or chemotherapy (pre-taxane).

In one embodiment, the subject has asymptomatic non-metastatic disease with negative scans for measurable disease and without rising PSA.

In one embodiment, subject has metastatic disease with positive scans for metastatic disease but without rising PSA and has not been treated with androgen-deprivation therapy or chemotherapy (pre-taxane).

In one embodiment, the subject has metastatic disease with positive scans for metastatic disease but without rising PSA, and has not been treated with abiraterone, enzalutamide, or apalutamide, or chemotherapy (pre-taxane).

In one embodiment, the subject has metastatic disease and has been treated with abiraterone, enzalutamide, or apalutamide, but has not received chemotherapy (pre-taxane).

In one embodiment, the concomitant treatment by androgen-deprivation therapy with a compound selected from 1-benzyl-6-(3,5-dimethylisoxazol-4-yl)-N-methyl-1H-imidazo[4,5-b]pyridin-2-amine (Compound I) and 1-benzyl-6-(3,5-dimethylisoxazol-4-yl)-1H-imidazo[4,5-b]pyridin-2-amine and pharmaceutically acceptable salts or co-crystals thereof to a subject that not previously has received chemotherapy (pre-taxane) demonstrate an unexpected superior safety profile by lacking thrombocytopenia as a dose limiting toxicity.

In one embodiment, the subject has metastatic disease and has been treated with abiraterone, enzalutamide, or apalutamide, but has not received chemotherapy (pre-taxane), for which treatment with another androgen-deprivation therapy is not recommended.

In one embodiment, the subject has metastatic disease and has been treated with androgen-deprivation therapy and chemotherapy.

In some embodiments, the subject is a human.

In some embodiments, the BET bromodomain inhibitor as described herein is administered concomitantly with another therapeutic agent and optionally further in combination with an immune checkpoint inhibitor. “Concomitantly” as used herein means that the BET bromodomain inhibitor and the other therapeutic agent are administered with a time separation of a few seconds (for example 15 sec., 30 sec., 45 sec., 60 sec. or less), several minutes (for example 1 min., 2 min., 5 min. or less, 10 min. or less, 15 min. or less), or 1-8 hours. When administered concomitantly, the BET bromodomain inhibitor and the other therapeutic agent may be administered in two or more administrations, and contained in separate compositions or dosage forms, which may be contained in the same or different package or packages.

In certain embodiments, the BET bromodomain inhibitor administered in the combination therapy of the invention is selected from Compound I and 1-benzyl-6-(3,5-dimethylisoxazol-4-yl)-1H-imidazo[4,5-b]pyridin-2-amine and is administered to a subject at a dose of 25 to 200 mg/day. In some embodiments, the compound selected from Compound I and 1-benzyl-6-(3,5-dimethylisoxazol-4-yl)-1H-imidazo[4,5-b]pyridin-2-amine is administered to a subject at a dose of 36 to 144 mg/day. In some embodiments, the compound selected from Compound I and 1-benzyl-6-(3,5-dimethylisoxazol-4-yl)-1H-imidazo[4,5-b]pyridin-2-amine for use in the combination therapies of the invention is administered to a subject at a dose of 48 mg to 120 mg/day. In some embodiments, the compound selected from Compound I and 1-benzyl-6-(3,5-dimethylisoxazol-4-yl)-1H-imidazo[4,5-b]pyridin-2-amine for use in the combination therapies of the invention is administered to a subject at a dose of 48 mg, 60 mg, 72 mg, 96 mg, or 120 mg/day. In any of the embodiments described herein, the compound selected from Compound I and 1-benzyl-6-(3,5-dimethylisoxazol-4-yl)-1H-imidazo[4,5-b]pyridin-2-amine may be administered in combination with 80 mg to 160 mg of enzalutamide. In any of the embodiments described herein, the compound selected from Compound I and 1-benzyl-6-(3,5-dimethylisoxazol-4-yl)-1H-imidazo[4,5-b]pyridin-2-amine may be administered in combination with 80 mg, 120 mg, or 160 mg of enzalutamide. In any of the embodiments described herein, the compound selected from Compound I and 1-benzyl-6-(3,5-dimethylisoxazol-4-yl)-1H-imidazo[4,5-b]pyridin-2-amine may be administered in combination with 500 mg to 1,000 mg of abiraterone. In any of the embodiments described herein, the compound selected from Compound I and 1-benzyl-6-(3,5-dimethylisoxazol-4-yl)-1H-imidazo[4,5-b]pyridin-2-amine may be administered in combination with 500 mg, 750 mg, or 1,000 mg of abiraterone. In any of the embodiments described herein, the compound selected from Compound I and 1-benzyl-6-(3,5-dimethylisoxazol-4-yl)-1H-imidazo[4,5-b]pyridin-2-amine may be administered in combination with 120 mg to 240 mg of apalutamide. In any of the embodiments described herein, the compound selected from Compound I and 1-benzyl-6-(3,5-dimethylisoxazol-4-yl)-1H-imidazo[4,5-b]pyridin-2-amine may be administered in combination with 120 mg or 180 mg, or 240 mg of apalutamide. In any of the embodiments described herein, the compound selected from Compound I and 1-benzyl-6-(3,5-dimethylisoxazol-4-yl)-1H-imidazo[4,5-b]pyridin-2-amine may be administered in combination with 100 mg to 300 mg twice-daily of darolutamide. In some embodiments, 36 to 144 mg of Compound I is administered in combination with 80 mg to 160 mg of enzalutamide, 500 mg to 1,000 mg of abiraterone, 120 mg to 240 mg of apalutamide, or 100 mg to 300 mg twice-daily of darolutamide.

In certain embodiments, the BET bromodomain inhibitor administered in the combination therapy of the invention is selected from pharmaceutically acceptable salts or co crystals of Compound I and 1-benzyl-6-(3,5-dimethylisoxazol-4-yl)-1H-imidazo[4,5-b]pyridin-2-amine and is administered to a subject at a dosage level providing an exposure in humans similar to an amount of 25 to 200 mg/day of the corresponding free base. In certain embodiments, the compound selected from pharmaceutically acceptable salts or co crystals of Compound I and 1-benzyl-6-(3,5-dimethylisoxazol-4-yl)-1H-imidazo[4,5-b]pyridin-2-amine may be administered in the combination therapies of the invention at a dosage level providing an exposure in humans similar to an amount of 36 to 144 mg/day of the corresponding free base. In certain embodiments, a compound selected from pharmaceutically acceptable salts or co crystals of Compound I and 1-benzyl-6-(3,5-dimethylisoxazol-4-yl)-1H-imidazo[4,5-b]pyridin-2-amine may be administered in the combination therapies of the invention at a dosage level providing an exposure in humans similar to an amount of 48 mg to 96 mg/day of the corresponding free base. In any of the embodiments described herein, the compound selected from pharmaceutically acceptable salts or co crystals of Compound I and 1-benzyl-6-(3,5-dimethylisoxazol-4-yl)-1H-imidazo[4,5-b]pyridin-2-amine may be administered in combination with 80 mg to 160 mg of enzalutamide, 500 mg to 1,000 mg of abiraterone, or 120 mg to 240 mg of apalutamide.

In certain embodiments, the subject has an activation of the ETS transcription factor family, either through activating mutations and/or translocations, including TMPRSS2-ERG, SLC45A3-ERG, NDRG1-ERG, DUX4-ERG, ELF4-ERG, ELK4-ERG, BZW2-ERG, CIDEC-ERG, DYRK1A-ERG, EWSR1-ERG, FUS-ERG, GMPR-ERG, HERPUD1-ERG, KCNJ6-ERG, ZNRF3-ERG, ETS2-ERG, ETV1-ERG, HNRNPH1-ERG, PAK1-ERG, PRKAB2-ERG, SMG6-ERG, SLC45A3-FL11, TMPRSS2-ETV1, SLC45A3-ETV1, FOXP1-ETV1, EST14-ETV1, HERVk17-ETV1, ERVK-24-ETV1, C150RF21-ETV1, HNRPA2B1-ETV1, ACSL3-ETV1, OR51E2-ETV1, ETV1 S100R, RBM25-ETV1, ACPP-ETV1, BMPR1B-ETV1, CANT1-ETV1, ERO1A-ETV1, CPED1-ETV1, HMGN2P46-ETV1, HNRNPA2B1-ETV1, SMG6-ETV1, FUBP1-ETV1, KLK2-ETV1, MIPOL1-ETV1, SLC30A4-ETV1, EWSR1-ETV1, TMPRSS2-ETV4, KLK2-ETV4, CANT1-ETV4, DDX5-ETV4, UBTF-ETV4, DHX8-ETV4, CCL16-ETV4, EDIL3-ETV4, EWSR1-ETV4, SLC45A3-ETV4, UBTF-ETV4, XPO7-ETV4, TMPRSS2-ETV5, SLC45A3-ETV5, ACTN4-ETV5, EPG5-ETV5, LOC284889-ETV5, RNF213-ETV5, SLC45A3-ELK4.

In certain embodiments, the subject has an activation of the ETS transcription factor family, either through activating mutations and/or translocations, including in certain embodiments, the subject has an activation of TMPRSS2-ERG, an ETS transcription factor family member, either through activating mutations and/or translocations.

In certain embodiments, the subject has less than 2.5 fold increase in PSA at 12 weeks of treatment.

In certain embodiments, the subject has at least a 2 fold decrease in PSA at 12 weeks of treatment.

In certain embodiments, the subject has a spike in PSA either at 4 weeks or 8 weeks of treatment. A spike at 4 weeks being defined as an increase in PSA at 4 weeks of treatment compared to the start of treatment with Compound I (Week 0), followed by a decrease in PSA from week 4 to week 8 of treatment. A spike at 8 weeks being defined as an increase in PSA at 8 weeks of treatment compared to 4 weeks of treatment (Week 4) followed by a decrease in PSA from week 8 to week 12 of treatment.

### REFERENCES

Aggarwal, R., Zhang, T., Small, E. J. & Armstrong, A. J. (2014) Neuroendocrine prostate cancer: subtypes, biology, and clinical outcomes. J Natl Compr Canc Netw, 12(5), 719-26.
Antonarakis, E. S., Lu, C., Wang, H., Luber, B., Nakazawa, M., Roeser, J. C., Chen, Y., Mohammad, T. A., Chen, Y., Fedor, H. L., Lotan, T. L., Zheng, Q., De Marzo, A. M., Isaacs, J. T., Isaacs, W. B., Nadal, R., Paller, C. J., Denmeade, S. R., Carducci, M. A., Eisenberger, M. A. & Luo, J. (2014) AR-V7 and resistance to enzalutamide and abiraterone in prostate cancer. N Engl J Med, 371(11), 1028-38.
Arora, V. K., Schenkein, E., Murali, R., Subudhi, S. K., Wongvipat, J., Balbas, M. D., Shah, N., Cai, L., Efstathiou, E., Logothetis, C., Zheng, D. & Sawyers, C. L. (2013) Glucocorticoid receptor confers resistance to antiandrogens by bypassing androgen receptor blockade. Cell, 155(6), 1309-22.
Asangani, I. A., Dommeti, V. L., Wang, X., Malik, R., Cieslik, M., Yang, R., Escara-Wilke, J., Wilder-Romans, K., Dhanireddy, S., Engelke, C., lyer, M. K., Jing, X., Wu, Y. M., Cao, X., Qin, Z. S., Wang, S., Feng, F. Y. & Chinnaiyan, A. M. (2014) Therapeutic targeting of BET bromodomain proteins in castration-resistant prostate cancer. Nature, 510(7504), 278-82.
Asangani, I. A., Wilder-Romans, K., Dommeti, V. L., Krishnamurthy, P. M., Apel, I. J., Escara-Wilke, J., Plymate, S. R., Navone, N. M., Wang, S., Feng, F. Y. & Chinnaiyan, A. M. (2016) BET Bromodomain Inhibitors Enhance Efficacy and Disrupt Resistance to AR Antagonists in the Treatment of Prostate Cancer. Mol Cancer Res, 14(4), 324-31.
Azad, A. A., Volik, S. V., Wyatt, A. W., Haegert, A., Le Bihan, S., Bell, R. H., Anderson, S. A., McConeghy, B., Shukin, R., Bazov, J., Youngren, J., Paris, P., Thomas, G., Small, E. J., Wang, Y., Gleave, M. E., Collins, C. C. & Chi, K. N. (2015b) Androgen receptor gene aberrations in circulating cell-free DNA: biomarkers of therapeutic resistance in castration-resistant prostate cancer. Clin Cancer Res, 21(10), 2315-24.
Beltran, H., Tomlins, S., Aparicio, A., Arora, V., Rickman, D., Ayala, G., Huang, J., True, L., Gleave, M. E., Soule, H., Logothetis, C. & Rubin, M. A. (2014) Aggressive variants of castration-resistant prostate cancer. Clin Cancer Res, 20(11), 2846-50.
Bianchini, D., Lorente, D., Rodriguez-Vida, A., Omlin, A., Pezaro, C., Ferraldeschi, R., Zivi, A., Attard, G., Chowdhury, S. & de Bono, J. S. (2014) Antitumour activity of enzalutamide (MDV3100) in patients with metastatic castration-resistant prostate cancer (CRPC) pre-treated with docetaxel and abiraterone. Eur J Cancer, 50(1), 78-84.
Ceribelli, M., Kelly, P. N., Shaffer, A. L., Wright, G. W., Xiao, W., Yang, Y., Mathews Griner, L. A., Guha, R., Shinn, P., Keller, J. M., Liu, D., Patel, P. R., Ferrer, M., Joshi, S., Nerle, S., Sandy, P., Normant, E., Thomas, C. J. & Staudt, L. M. (2014) Blockade of oncogenic IkappaB kinase activity in diffuse large B-cell lymphoma by bromodomain and extraterminal domain protein inhibitors. Proc Natl Acad Sci USA, 111(31), 11365-70.
Chan, S. C., Seith, L. A., Li, Y., Nyquist, M. D., Miao, L., Bradner, J. E., Raj, G. V., Tilley, W. D. & Dehm, S. M. (2015) Targeting chromatin binding regulation of constitutively active AR variants to overcome prostate cancer resistance to endocrine-based therapies. Nucleic Acids Res, 43(12), 5880-97.
Chou, T. C. and Talalay, P., (1984) Quantitative analysis of dose-effect relationships: The combined effects of multiple drugs or enzyme inhibitors, Advances in Enzyme Regulation 22, 27-55.
Dang, Q., Li, L., Xie, H., He, D., Chen, J., Song, W., Chang, L. S., Chang, H. C., Yeh, S. & Chang, C. (2015) Anti-androgen enzalutamide enhances prostate cancer neuroendocrine (NE) differentiation via altering the infiltrated mast cells-->androgen receptor (AR)-->miRNA32 signals. Mol Oncol, 9(7), 1241-51.
Gallagher, S. J., Mijatov, B., Gunatilake, D., Gowrishankar, K., Tiffen, J., James, W., Jin, L., Pupo, G., Cullinane, C., McArthur, G. A., Tummino, P. J., Rizos, H. & Hersey, P. (2014) Control of NF-κB activity in human melanoma by bromodomain and extra-terminal protein inhibitor I-BET151. Pigment Cell Melanoma Res, 27(6), 1126-37.
Gao, L., Schwartzman, J., Gibbs, A., Lisac, R., Kleinschmidt, R., Wilmot, B., Bottomly, D., Coleman, I., Nelson, P., McWeeney, S. & Alumkal, J. (2013) Androgen receptor promotes ligand-independent prostate cancer progression through c-Myc upregulation. PloS one, 8(5), e63563.
Isikbay, M., Otto, K., Kregel, S., Kach, J., Cai, Y., Vander Griend, D. J., Conzen, S. D. & Szmulewitz, R. Z. (2014) Glucocorticoid receptor activity contributes to resistance to androgen-targeted therapy in prostate cancer. Horm Cancer, 5(2), 72-89.
Jin, R., Sterling, J. A., Edwards, J. R., DeGraff, D. J., Lee, C., Park, S. I. & Matusik, R. J. (2013) Activation of NF-kappa B signaling promotes growth of prostate cancer cells in bone. PloS one, 8(4), e60983.
Lamb, A. D., Massie, C. E. & Neal, D. E. (2014) The transcriptional programme of the androgen receptor (AR) in prostate cancer. BJU Int, 113(3), 358-66.
Loriot, Y., Bianchini, D., Ileana, E., Sandhu, S., Patrikidou, A., Pezaro, C., Albiges, L., Attard, G., Fizazi, K., De Bono, J. S. & Massard, C. (2013) Antitumour activity of abiraterone acetate against metastatic castration-resistant prostate cancer progressing after docetaxel and enzalutamide (MDV3100). Ann Oncol, 24(7), 1807-12.
Nadiminty, N., Tummala, R., Liu, C., Yang, J., Lou, W., Evans, C. P. & Gao, A. C. (2013) NF-kappaB2/p52 induces resistance to enzalutamide in prostate cancer: role of androgen receptor and its variants. Mol Cancer Ther, 12(8), 1629-37.
Nakazawa, M., Antonarakis, E. S. & Luo, J. (2014) Androgen receptor splice variants in the era of enzalutamide and abiraterone. Horm Cancer, 5(5), 265-73.
Noonan, K. L., North, S., Bitting, R. L., Armstrong, A. J., Ellard, S. L. & Chi, K. N. (2013) Clinical activity of abiraterone acetate in patients with metastatic castration-resistant prostate cancer progressing after enzalutamide. Ann Oncol, 24(7), 1802-7.
Schrader, A. J., Boegemann, M., Ohlmann, C. H., Schnoeller, T. J., Krabbe, L. M., Hajili, T., Jentzmik, F., Stoeckle, M., Schrader, M., Herrmann, E. & Cronauer, M. V. (2014) Enzalutamide in castration-resistant prostate cancer patients progressing after docetaxel and abiraterone. Eur Urol, 65(1), 30-6.
Scher, H. I., Halabi, S., Tannock, I., Morris, M., Sternberg, C. N., Cardussi, M. A., Eisenberger, M. A., Higano, C., Bubley, G. J., Dreicer, R., Petrylak, D., Kantoff, P., Basch, E., Kelly, W. K., Figg, W. D., Small, E. J., Beer, T. M., Wilding, G., Martin, A., Hussain, M. (2008) Design and End Point of Clinical Trials for Patients with Progressive Prostate Cancer and Castrate Levels of Testosterone: Recommendations of the Prostate Cancer Clinical Trials Working Group. J. Clin. Onco. 26, 1148-1159.
Shah, N., Wang, P., Wongvipat, J., Karthaus, W. R., Abida, W., Armenia, J., Rockowitz, S., Drier, Y., Bernstein, B. E., Long, H. W., Freedman, M. L., Arora, V. K., Zheng, D. & Sawyers, C. L. (2017) Regulation of the glucocorticoid receptor via a BET-dependent enhancer drives antiandrogen resistance in prostate cancer. Elife, 6.
Welti, J., Sharp, A., Yuan, W., Dolling, D., Nava Rodrigues, D., Figueiredo, I., Gil, V., Neeb, A., Clarke, M., Seed, G., et al. (2018) Targeting Bromodomain and Extra-Terminal (BET) Family Proteins in Castration-Resistant Prostate Cancer (CRPC). Clinical cancer research: an official journal of the American Association for Cancer Research 24, 3149-3162.
Wyatt, A. W. & Gleave, M. E. (2015) Targeting the adaptive molecular landscape of castration-resistant prostate cancer. EMBO Mol Med, 7(7), 878-94.
Wyce, A., Degenhardt, Y., Bai, Y., Le, B., Korenchuk, S., Crouthame, M. C., McHugh, C. F., Vessella, R., Creasy, C. L., Tummino, P. J. & Barbash, O. (2013) Inhibition of BET bromodomain proteins as a therapeutic approach in prostate cancer. Oncotarget, 4(12), 2419-29.
Zeng, W., Sun, H., Meng, F., Liu, Z., Xiong, J., Zhou, S., Li, F., Hu, J., Hu, Z. & Liu, Z. (2015) Nuclear C-MYC expression level is associated with disease progression and potentially predictive of two year overall survival in prostate cancer. Int J Clin Exp Pathol, 8(2), 1878-88.
Zou, Z., Huang, B., Wu, X., Zhang, H., Qi, J., Bradner, J., Nair, S. & Chen, L. F. (2014) Brd4 maintains constitutively active NF-kappaB in cancer cells by binding to acetylated ReIA. Oncogene, 33(18), 2395-404.

### EXAMPLES

Tissue culture media and reagents were obtained from ThermoFisher Scientific. Enzalutamide, apalutamide, abiraterone acetate, and darolutamide were obtained from Selleck Chemicals. Metribolone (R1881) from Toronto Research Chemicals.

### Example 1: Synthesis of Compound I

### Step A: Synthesis of 5-bromo-N³-(phenylmethylene)pyridine-2,3-diamine (Compound B)

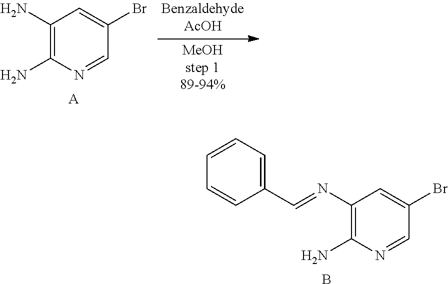

Starting material A was dissolved in methanol and acetic acid. The solution was cooled to 0-5° C. and benzaldehyde was added dropwise. Once the reaction was complete, process water and a NaHCO₃ solution was added dropwise, keeping the temperature low (0-5° C.). The solid was filtered off and washed with methanol/water 1:1, followed by drying, leaving Compound B in 94% yield and +99% purity by HPLC. 1H-NMR (DMSO-d₆): δ 8.75 (1H), 8.04 (2H), 7.93 (1H), 7.65 (1H), 7.50-7.60 (3H).

### Step B: Synthesis of N³-benzyl-5-bromopyridine-2,3-diamine (Compound C)

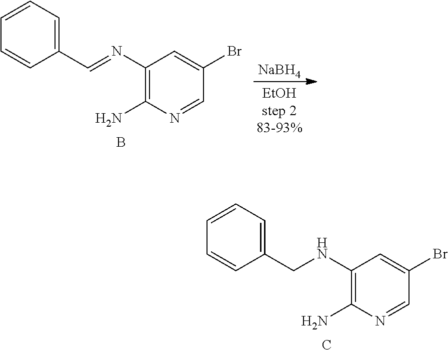

Compound B was dissolved in ethanol and NaHB₄ was added in portions keeping the temperature between 15-25° C. The reaction mixture was stirred for 8-15 h until the reaction was complete as monitored by HPLC. A HCl solution was added, adjusting pH to 6-7, followed by process water, keeping the temperature between 15-25° C. The mixture was stirred for 1-5 h, filtered and washed with an ethanol/water mixture. Following drying at ˜60° C. for 15-20 h, Compound C was obtained. 1H-NMR (DMSO-d₆): δ 7.2-7.4 (6H), 6.55 (1H), 5.70-5.83 (3H), 4.30 (2H).

### Step C: Synthesis of N³-benzyl-5-(3,5-dimethyl-1,2-oxazol-4-yl)pyridine-2,3-diamine (Compound D)

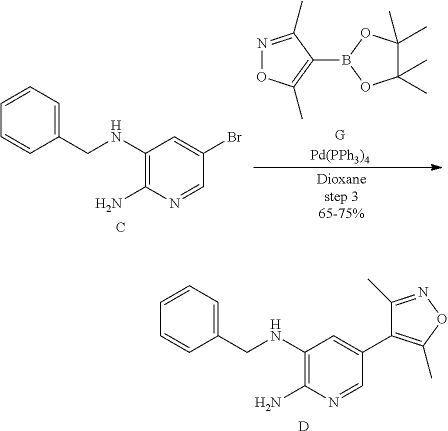

Compound C, Compound G, and potassium phosphate tribasic trihydrate were mixed followed by addition of 1,4-Dioxane and process water. The resulting mixture was thoroughly purged with nitrogen. Tetrakis(triphenylphosphine)palladium(0) was added and the mixture was heated to ≥90° C. until the ratio of Compound C to Compound D was not more than 1%. After cooling, the reaction mixture was filtered, the solid washed with 1,4-dioxane and then concentrated. Process water was added and the mixture was stirred until the amount of Compound D remaining in the mother liquors was not more than 0.5%. Compound D was isolated by filtration and sequentially washed with 1,4-dioxane/water and t-butylmethyl ether. The wet cake was mixed in methylene chloride and silica gel. After stirring, the mixture was filtered then concentrated. The mixture was cooled and t-butylmethyl ether was added. The product was isolated by filtration and dried until the methylene chloride, t-butylmethyl ether, and moisture levels are not more than 0.5%. ¹H-NMR (DMSO-d₆): δ 7.30-7.45 (4H), 7.20-7.25 (2H), 6.35 (1H), 5.65-5.80 (3H), 4.30-4.40 (2H), 2.15 (3H), 1.95 (3H).

### Step D: Synthesis of 1-benzyl-6-(3,5-dimethyl-1,2-oxazol-4-yl)-3H-imidazo[4,5-b]pyridin-2-one (Compound E)

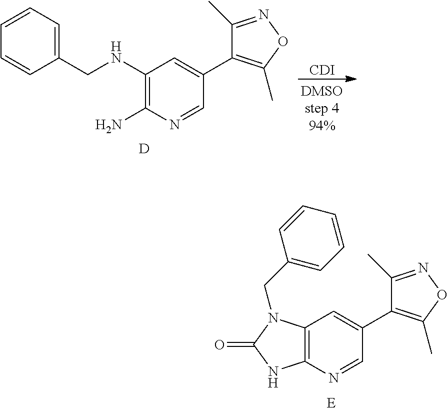

Carbonyldiimidazole solid was added to a stirring mixture of Compound D and dimethylsulfoxide. The mixture was heated until the ratio of Compound D to Compound E was NMT 0.5%. The mixture was cooled and process water was added over several hours. The resulting mixture was stirred at ambient temperature for at least 2 h. The product was isolated by filtration and washed with process water. The dimethylsulfoxide was verified to be NMT 0.5% before drying using heat and vacuum. Drying was complete when the moisture level was NMT 0.5%, leaving Compound E. ¹H-NMR (DMSO-d₆): δ 11.85 (1H), 7.90 (1H), 7.20-7.45 (6H), 5.05 (2H), 3.57 (3H), 2.35 (3H), 2.15 (3H).

### Step E: Synthesis of 4-[1-benzyl-2-chloro-1H-imidazo[4,5-b]pyridine-6-yl]-3,5-dimethyl-1,2-oxazole (Compound F)

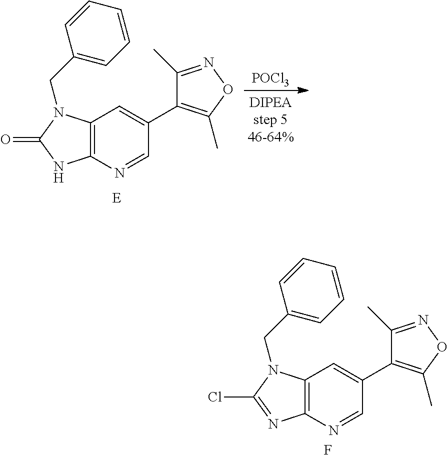

Compound E and phosphorus oxychloride were mixed and then treated with diisopropylethyl amine (DIPEA), which can be added dropwise. The resulting mixture was heated for several hours, cooled, and sampled for reaction completion. If the ratio of Compound E to Compound F was not more than 0.5% then the reaction was complete. Otherwise, the reaction was heated for additional time and sampled as before. After the reaction was complete, the mixture was concentrated then cooled. Ethyl acetate was added and the mixture was concentrated under vacuum several times. Ethyl acetate (EtOAc) was added to the concentrate, the mixture was cooled and then added to aqueous sodium bicarbonate. The organic phase was separated and the organic layer was washed with aqueous sodium bicarbonate and then water. The organic phase was concentrated, ethyl acetate was added, and the mixture was concentrated to assure that the moisture level was not more than 0.2%. The mixture in ethyl acetate was decolorized with carbon. The mixture was concentrated and n-heptane was added. The product was isolated by filtration and dried under vacuum. Drying was complete when residual moisture, ethyl acetate, and n-heptane were not more than 0.5%. ¹H-NMR (MeOH-d₄): δ 8.40 (1H), 7.90 (1H), 7.25-7.45 (5H), 5.65 (2H), 2.37 (3H), 2.22 (3H).

### Step F: Synthesis of 1-benzyl-6-(3,5-dimethyl-1,2-oxazol-4-yl)-N-methyl-1H-imidazo[4,5-b]pyridine-2-amine (Compound 1)

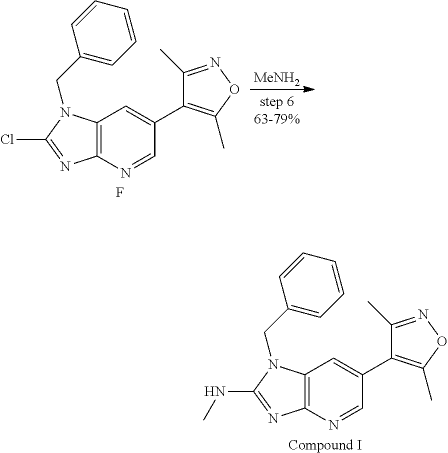

Compound F was mixed with methylamine in tetrahydrofuran (THF) and stirred at ambient temperature until the ratio of Compound F to Compound I was NMT 0.1% by HPLC. After reaction completion, the mixture was concentrated under vacuum, process water added, and the product isolated by filtration. The filter cake was washed with process water. The wet cake was dissolved in hydrochloric acid and the resulting solution was washed with methylene chloride to remove impurities. The aqueous solution was neutralized with a sodium hydroxide solution and Compound I was isolated by filtration, washed with process water, and dried under vacuum. If necessary, to remove any remaining hydrochloric acid, the dried material can be dissolved in ethanol, treated with a solution of sodium hydroxide in ethanol, followed by addition of process water to precipitate the product. Compound I was isolated by filtration, washed with process water, and dried. ¹H-NMR (DMSO-d₆): δ 7.96 (d, 1H, J=2.0 Hz), 7.42 (d, 1H, J=2.0 Hz), 7.37 (q, 1H, J=4.2 Hz), 7.32 (m, 2H), 7.26 (m, 1H), 7.24 (m, 2H), 5.30 (s, 2H), 3.00 (d, 3H, 4.5 Hz), 2.34 (s, 3H), 2.16 (s, 3H). ¹³C-NMR (DMSO-d₆): δ 164.8, 158.4, 157.7, 156.0, 141.1, 136.4, 128.6 (2C), 127.5, 127.4, 127.2 (2C), 115.8, 114.2 (2C), 44.5, 29.3, 11.2, 10.3.

### Example 2: Crystalline Mesylate of Compound I

About 5 g of Compound I was dissolved in ethanol (115 mL) and a solution of methanesulfonic acid in ethanol (10 mL, 158.7 mg/mL) was added, according to a 1:1 molar ratio. The mixture was shaken at 50° C. for 2 h before concentrated to half volume and stirred overnight. The formed solid (mesylate salt/co-crystal of Compound I Form 1) was isolated, dried, and characterized.

The mesylate salt/co crystal of Compound I Form I was also obtained from other solvents and solvent mixtures, including acetone and acetonitrile.

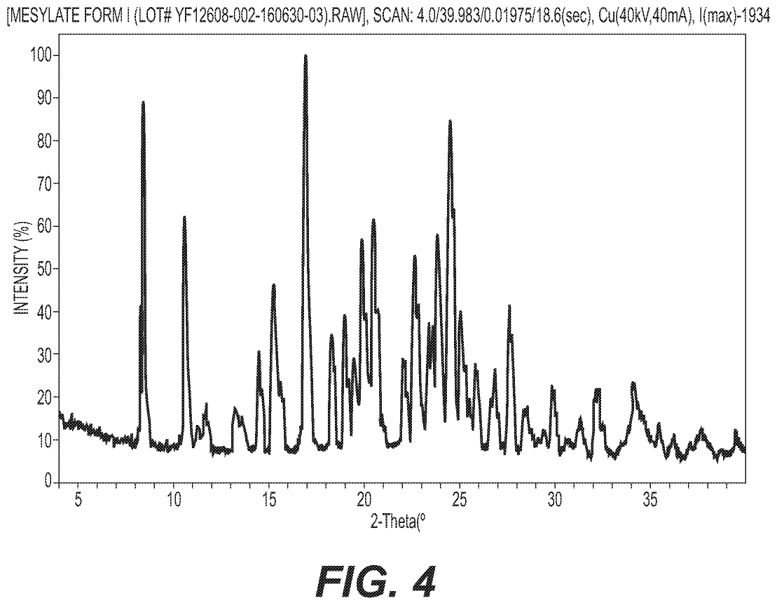

The mesylate salt/co crystal of Compound I Form I was characterized by XRPD comprising the following peaks, in terms of 2-theta, at 8.4±0.2, 10.6 0.2, 11.7 0.2, 14.5 0.2, 15.3±0.2, 16.9±0.2, 18.2±0.2, 19.0±0.2, 19.9±0.2, 20.5±0.2, 22.6±0.2, 23.8±0.2, 24.5±0.2, and 27.6±0.2 degrees, as determined on a diffractometer using Cu-K_{α} radiation tube (FIG. 4).

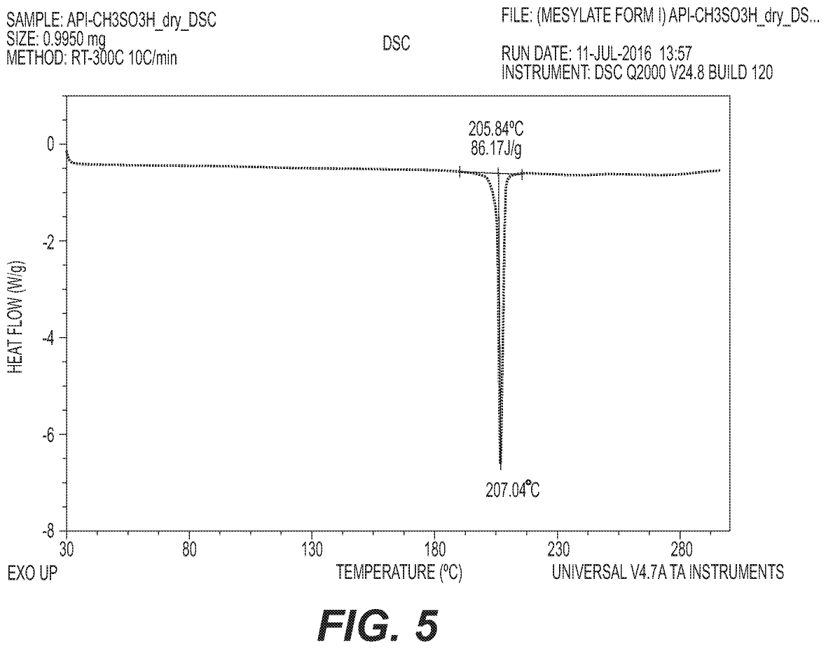

The mesylate salt/co crystal of Compound I Form I was characterized by DSC having an endothermic peak at a temperature of about 207° C. (FIG. 5).

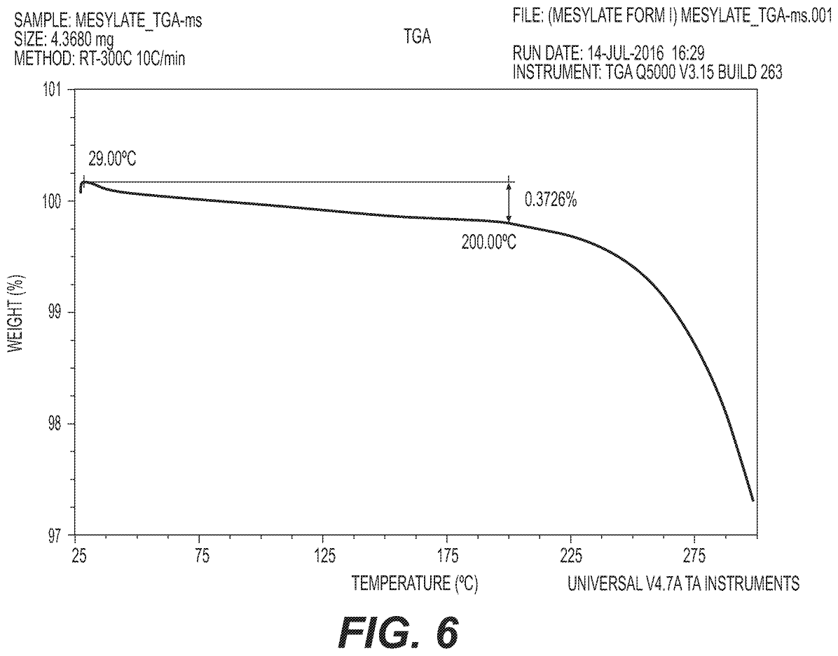

The mesylate salt/co crystal of Compound I Form I was characterized by TGA, having a thermogram as shown in FIG. 6, confirming that Compound I Form I is an anhydrous form.

### Example 3: Synergistic Inhibition of VCaP Cell Viability by Combination of Compound I with Enzalutamide

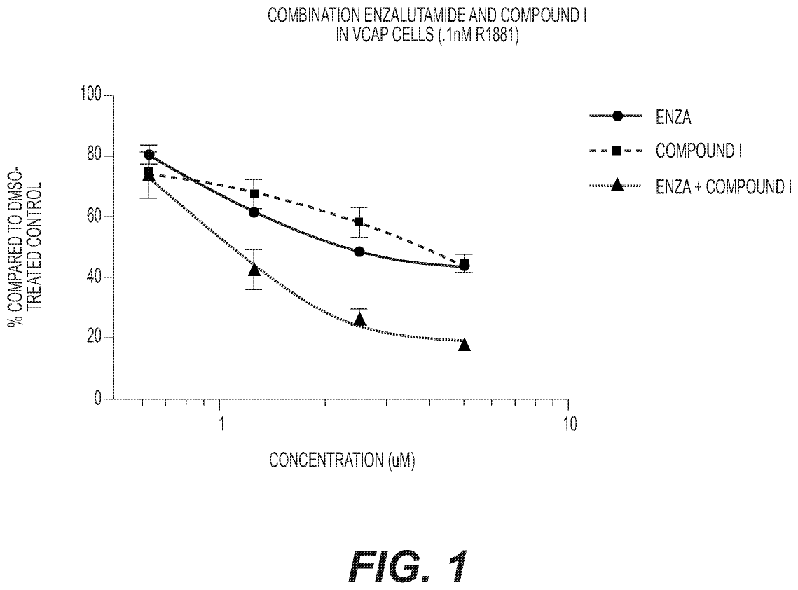

VCaP cells (CRL-2876) were plated at a density of 10,000 cells per well in 96 well flat bottom plates in D-MEM media containing 10% charcoal-stripped FBS and penicillin/streptomycin and incubated for 24 hours at 37° C., 5% CO₂. Media was replaced with D-MEM containing 10% charcoal-stripped FBS with 0.1 nM R1881 treated with constant ratios of either Compound I or enzalutamide as single agents, or a combination of both drugs at four different concentrations (2×IC50, 1×IC50, 0.5×IC50, 0.25×IC50), and incubated at 37° C., 5% CO₂ for 3 to 7 days. If cells were incubated for 7 days, they were retreated as described above on the 3^{rd} or 4^{th} day. If cells were incubated for 7 days, they were retreated as described above on the 3^{rd} or 4^{th} day. Triplicate wells were used for each concentration and wells containing only media with 0.1% DMSO were used as a control. To measure cell viability, 100 uL of a 1:100 dilution of GF-AFC substrate into the Assay Buffer (CellTiter Fluor Cell Viability Assay (Promega)) were added to each well and incubated at 37° C., 5% CO₂ for an additional 30-90 minutes. Fluorescence at 380-400 nm Excitation/505 nm Emission was read in a fluorometer and the percentage of cell titer relative to DMSO-treated cells was calculated after correcting for background by subtracting the blank well's signal. IC50 values for single agents were calculated using the GraphPad Prism software. Quantification of synergy was done by calculating combination indices (CI) using the CalcuSyn software (Biosoft) based on the Chou-Talalay algorithm (Chou and Talalay, 1984), and averaging the Cl values for the effective doses (ED) 50, 75, and 90. As shown in FIG. 1, addition of Compound I to enzalutamide resulted in improved inhibition of cell viability compared to either single agent with an average Cl value of 0.5.

### Example 4: Synergistic Inhibition of VCaP Cell Viability by Combination of Compound I with Apalutamide (ARN-509)

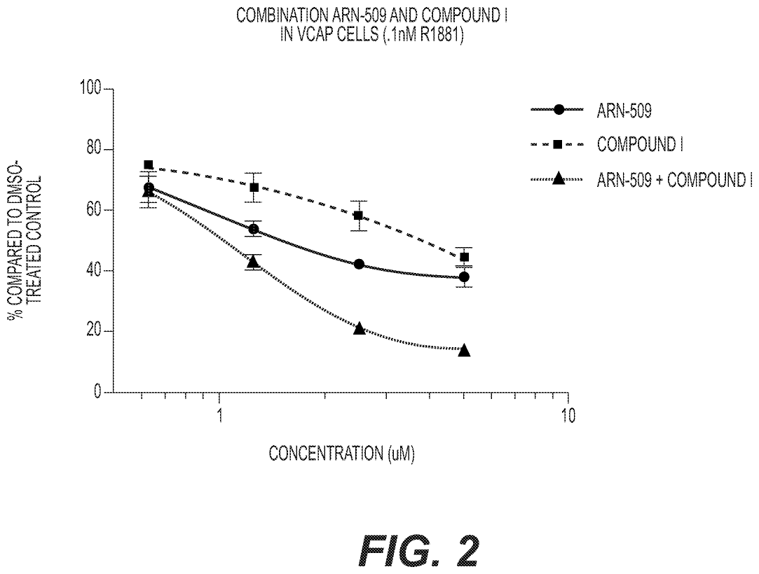

VCaP cells (CRL-2876) were plated at a density of 10,000 cells per well in 96 well flat bottom plates in D-MEM media containing 10% charcoal-stripped FBS and penicillin/streptomycin and incubated for 24 hours at 37° C., 5% CO₂. Media was replaced with D-MEM containing 10% charcoal-stripped FBS with 0.1 nM R1881 treated with constant ratios of either Compound I or apalutamide as single agents, or a combination of both drugs at four different concentrations (2×IC50, 1×IC50, 0.5×IC50, 0.25×IC50), and incubated at 37° C., 5% CO₂ for 3 to 7 days. If cells were incubated for 7 days, they were retreated as described above on the 3^{rd} or 4^{th} day. If cells were incubated for 7 days, they were retreated as described above on the 3^{rd} or 4^{th} day. Triplicate wells were used for each concentration and wells containing only media with 0.1% DMSO were used as a control. To measure cell viability, 100 uL of a 1:100 dilution of GF-AFC substrate into the Assay Buffer (CellTiter Fluor Cell Viability Assay (Promega)) were added to each well and incubated at 37° C., 5% CO₂ for an additional 30-90 minutes. Fluorescence at 380-400 nm Excitation/505 nm Emission was read in a fluorometer and the percentage of cell titer relative to DMSO-treated cells was calculated after correcting for background by subtracting the blank well's signal. IC50 values for single agents were calculated using the GraphPad Prism software. Quantification of synergy was done by calculating combination indices (CI) using the CalcuSyn software (Biosoft) based on the Chou-Talalay algorithm (Chou and Talalay, 1984), and averaging the Cl values for the effective doses (ED) 50, 75, and 90. As shown in FIG. 2, addition of Compound I to apalutamide resulted in improved inhibition of cell viability compared to either single agent with an average Cl value of 0.4.

### Example 5: Synergistic Inhibition of LAPC-4 Cell Viability by Combination of Compound I with Abiraterone Acetate

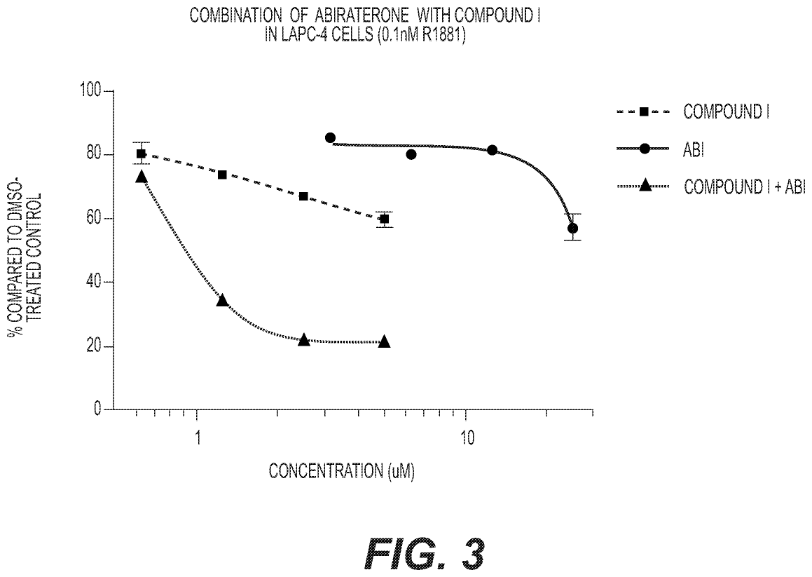

LAPC-4 cells (CRL-13009) were plated at a density of 5,000 cells per well in 96 well flat bottom plates in IMDM media containing 10% charcoal-stripped FBS and penicillin/streptomycin and incubated for 24 hours at 37° C., 5% CO₂. Media was replaced with IMDM containing 10% charcoal-stripped FBS with 1 nM R1881 treated with constant ratios of either Compound I or abiraterone acetate as single agents, or a combination of both drugs at four different concentrations (2×IC50, 1×IC50, 0.5×IC50, 0.25×IC50), and incubated at 37° C., 5% CO₂ for 3 to 7 days. Triplicate wells were used for each concentration and wells containing only media with 0.1% DMSO were used as a control. To measure cell viability, 100 uL of a 1:100 dilution of GF-AFC substrate into the Assay Buffer (CellTiter Fluor Cell Viability Assay (Promega)) were added to each well and incubated at 37° C., 5% CO₂ for an additional 30-90 minutes. Fluorescence at 380-400 nm Excitation/505 nm Emission was read in a fluorometer and the percentage of cell titer relative to DMSO-treated cells was calculated after correcting for background by subtracting the blank well's signal. IC50 values for single agents were calculated using the GraphPad Prism software. Quantification of synergy was done by calculating combination indices (CI) using the CalcuSyn software (Biosoft) based on the Chou-Talalay algorithm (Chou and Talalay, 1984), and averaging the CI values for the effective doses (ED) 50, 75, and 90. As shown in FIG. 3, addition of Compound I to abiraterone acetate resulted in improved inhibition of cell viability compared to either single agent with an average Cl value of 0.09.

### Example 6: Clinical Development

Compound I has been tested as a single agent and in combination with enzalutamide in humans with CRPC. Pharmaceutically acceptable salts of Compound I or a co-crystal thereof, particularly a mesylate salt/co-crystal of Compound I Form I, as well as other therapeutic agents such as, abiraterone, apalutamide, and darolutamide can be tested in the same manner.

A Phase 1b dose escalation study (3+3 design) has evaluated the pharmacokinetics, safety, tolerability, and target engagement of Compound I+enzalutamide. The dose escalation was tested up to a dose of 144 mg without reaching a maximum tolerated dose. Additional dose levels and dosing schedules could be explored to further define the maximal therapeutic efficacy. The target engagement was measured in a blood assay, and changes in the levels of mRNA were detected for a number of markers, including MYC, CCR1, IL1RN, GPR183, HEXIM1, PD-L1, IL-8, A2AR, TIM-3.
A Phase 2a dose confirmation study, evaluated Compound I at the 48 mg and 96 mg doses in combination with enzalutamide in subjects who are chemotherapy-naïve and progressed on enzalutamide and/or abiraterone. Pharmacokinetics, safety, tolerability, and target engagement, PSA response, as well as time to radiographic progression at a well-tolerated dose was used to determine a recommended Phase 2b dose. Subject blood and tumor samples has been molecularly profiled to determine responsive vs. non-responsive subjects to combination therapy and provides proof of mechanism.

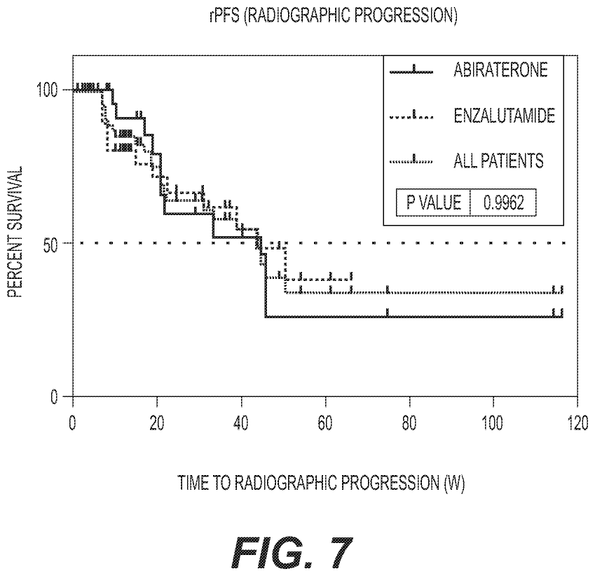

As shown in FIG. 7 and the table below, data evaluated from the Phase 2a study shows continued rPFS benefit for 2nd line mCRPC patients treated with Compound I+enzalutamide with an overall rPFS of 44.6 weeks compared to the expected 24-28 weeks for enzalutamide alone. Abiraterone and enzalutamide progressors showed similar benefit of the combination of Compound I with enzalutamide. Prolonged rPFS in patients with high and low tumor burden was also detected, including two partial responses, one in a patient previously progressing on abiraterone, and one progressing on prior enzalutamide. Two abiraterone progressors have a PSA90>117 weeks, and 7 patients with prior progression on enzalutamide received Compound I+enzalutamide >52 weeks.

| rPFS (Radio | Abiraterone | Enzalutamide | |
|---|---|---|---|
| only) | Progressors | Progressors | All patients |
| # of Patients | 30 | 45 | 75 |
| # of Events | 11 | 14 | 24 |
| Median PFS | 44.6 | 43.9 | 44.6 |
| (weeks) | | | |

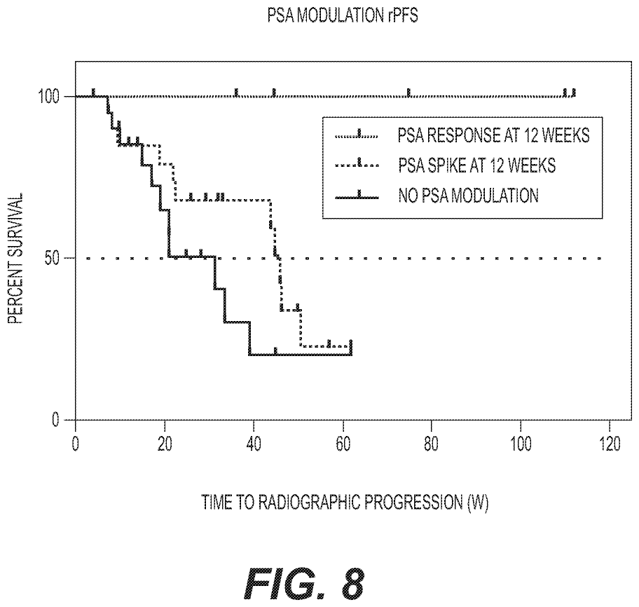

As shown in FIG. 8 and the table below, patients with a PSA response had a median radiation progression-free survival that was not yet reached at 120 weeks, and patients that had a PSA spike at either week 4 or week 8 had a median radiation progression-free survival of 45.9 weeks compared to patients that did not show such PSA spike or response whom had a median radiation progression free survival of 31.3 weeks. PSA response was defined as a decline of >50% of PSA at 12 weeks compared to the screening value. PSA spikes are defined in Example 7.

| rPFS (Radio | | PSA | No PSA |
|---|---|---|---|
| only) | PSA Spike | Response | Modulation |
| # of | 21 | 5 | 21 |
| Patients | | | |
| # of Events | 11 | 0 | 11 |
| Median PFS | 45.9 | Not yet | 31.3 |
| (weeks) | | reached | |

A randomized Phase 2b study will be used to confirm the phase 2 dose in a larger population, as well as identify sub-populations responding well to the combination therapy. A number of combinations of Compound I and another therapeutic agent can be explored.

A Phase 3 study will be a double blinded, randomized study of Compound I or a pharmaceutically acceptable salt or co-crystal thereof and another therapeutic agent (abiraterone, enzalutamide, darolutamide, or apalutamide) compared to placebo in subjects with CRPC. The primary end-point can be overall survival or time to radiographic progression.

### Example 7: PSA Spikes at 4 Weeks or 8 Weeks on Treatment with Compound I and Enzalutamide

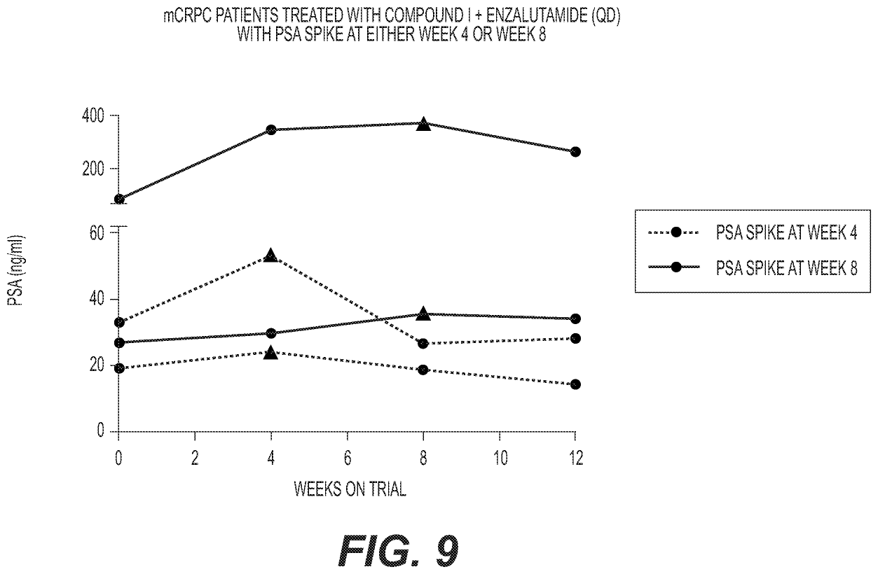

mCRPC patients with prior progression on abiraterone and/or enzalutamide were dosed QD with a combination of Compound I and enzalutamide. Several patients had a spike in PSA at either 4 weeks or 8 weeks post QD dosing with Compound I. FIG. 9 shows an example of 2 patients with a PSA spike at week 4, and 2 patients with a PSA spike at week 8. A spike at 4 weeks being defined as an increase in PSA at 4 weeks of treatment compared to the start of treatment (Week 0), followed by a decrease in PSA from week 4 to week 8 of treatment. A spike at 8 weeks being defined as an increase in PSA at 8 weeks of treatment compared to 4 weeks of treatment (Week 4) followed by a decrease in PSA from week 8 to week 12 of treatment. As shown in FIG. 8, subjects with PSA spikes had a longer radiation progression free survival compared to patients that did not have a PSA spike (45.9 vs. 31.3 weeks).

### Example 8: Distribution of ETS Mutations/Fusions and Response to the Combination of Compound I With Enzalutamide in mCRPC Patients

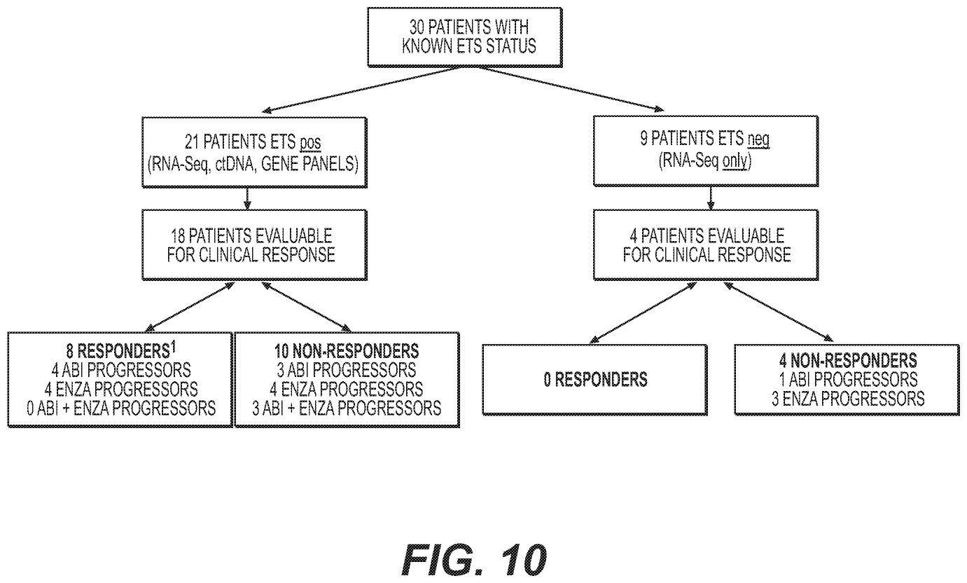

mCRPC patients with prior progression on abiraterone and/or enzalutamide were dosed QD with a combination of Compound I and enzalutamide. Patients with characterized mutations or fusions involving an ETS family member or the absence of such fusions or mutations and their response to the combination are depicted in FIG. 10. Responders are defined by >24 weeks post Compound I dosing without clinical or radiographic progression and Non-Responders by ≤24 weeks before radiographic or clinical progression. Patients with ETS mutations or fusions were similarly distributed between responders and non-responders, whereas there were no responders in patients that did not have an ETS mutation or fusion.

### Example 9: Distribution of ETS Mutations/Fusions, PSA Responses or Spikes, and Response to the Combination of Compound I with Enzalutamide in mCRPC Patients

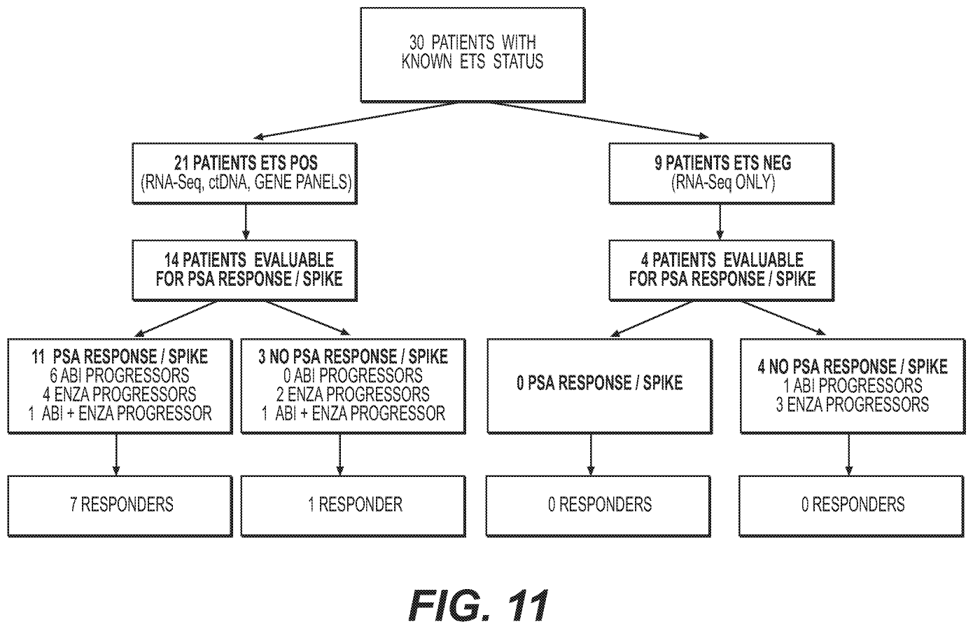

mCRPC patients with prior progression on abiraterone and/or enzalutamide were dosed QD with a combination of Compound I and enzalutamide. Patients with characterized mutations or fusions involving an ETS family member or the absence of such fusions or mutations and their response to the combination as well as the presence or absence of a PSA response or spike at either 4 or 8 weeks is depicted in FIG. 11. Responders are defined by >24 weeks post Compound I dosing without clinical or radiographic progression and Non-Responders by ≤24 weeks before radiographic or clinical progression. PSA response is defined by a decrease of ≥50% in the level of PSA at 12 weeks after the start of Dosing of Compound I. Presence of patients with ETS mutations or fusions was enriched in the patients with a PSA response or PSA spike at either 4 or 8 weeks.

### Example 10: Induction of the Immune Response and Interferon Gamma Signaling in the Tumor in Response to the Combination of Compound I with Enzalutamide in mCRPC Patients

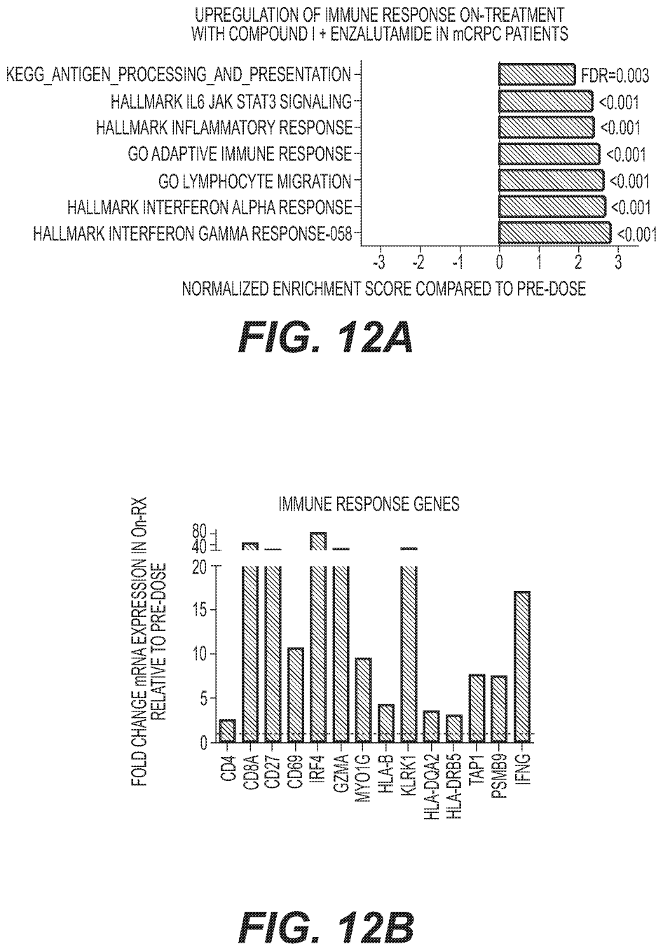

An mCRPC patient with prior progression on enzalutamide was dosed QD with f Compound I while continuing enzalutamide. A tumor biopsy was obtained at screening (on enzalutamide) and after 8 weeks (on enzalutamide and Compound 1) of dosing.l. Whole transcriptome (RNA-Seq) analysis was done on the two biopsies and alignment was done using the STAR software, and differential gene expression analysis with Cufflinks using the BaseSpace™ Sequence Hub default parameters between December 2018 and August 2019. Additional independent analysis was done using the SALMON alignment software and BioConductor. Identification of differentially expressed gene signatures was done using geneset enrichment analysis (GSEA) using gene signatures from the Molecular Signature Database (Subramanian A, Tamayo P, et al. (2005, PNAS 102, 15545-15550); Liberzon A, et al. (2011, Bionformatics 27, 1739-1740); Liberzon A, et al. (2015, Cell Systems 1, 417-425). As shown in FIG. 12A, several immune-related signatures were significantly up-regulated in the on-treatment biopsy. The relevant genesets are indicated in the figure and genes involved in each geneset can be downloaded from MSigDB. In FIG. 12B, some of the genes found in these genesets are graphed to show the extent of upregulation. Upregulation of genesets involved in adaptive immune response, antigen presentation, and interferon-gamma signaling suggests that the combination of Compound I and enzalutamide have induced an immunoresponsive phenotype, and thus that patients would respond to a triple combination of Compound I, enzalutamide, and a checkpoint inhibitor.

1. A method for treating a prostate cancer comprising administrating to a subject in need thereof a BET bromodomain inhibitor selected from 1-benzyl-6-(3,5-dimethylisoxazol-4-yl)-N-methyl-1H-imidazo[4,5-b]pyridin-2-amine (Compound I), 1-benzyl-6-(3,5-dimethylisoxazol-4-yl)-1H-imidazo[4,5-b]pyridin-2-amine, and pharmaceutically acceptable salts/co-crystals thereof, with a second therapeutic agent.

2. The method according to claim 1, wherein the BET bromodomain inhibitor is Compound I.

3. The method according to claim 1, wherein the BET bromodomain inhibitor is the mesylate salt/co-crystal of Compound I Form 1.

4. The method according to claim 1, wherein the second therapeutic agent is an androgen receptor antagonist.

5. The method according to claim 1, wherein the second therapeutic agent is an androgen synthesis inhibitor.

6. The method according to claim 1, wherein the second therapeutic agent is enzalutamide.

7. The method according to claim 1, wherein the second therapeutic agent is apalutamide.

8. The method according to claim 1, wherein the second therapeutic agent is abiraterone.

9. The method according to claim 1, wherein the prostate cancer is castration-resistant prostate cancer or metastatic castration-resistant prostate cancer.

10. The method according to claim 1, wherein the subject previously has been treated with a prostate cancer therapy.

11. The method according to claim 10, wherein the prostate cancer therapy is an androgen-deprivation therapy.

12. The method according to claim 1, wherein the subject previously has shown disease progression on androgen-deprivation therapy.

13. The method according to claim 1, wherein the subject has not previously been treated with androgen-deprivation therapy.

14. A method according to claim 11, wherein the androgen-deprivation therapy is enzalutamide, apalutamide, or abiraterone.

15. The method according to claim 1, wherein a compound selected from 1-benzyl-6-(3,5-dimethylisoxazol-4-yl)-N-methyl-1H-imidazo[4,5-b]pyridin-2-amine (Compound I) and 1-benzyl-6-(3,5-dimethylisoxazol-4-yl)-1H-imidazo[4,5-b]pyridin-2-amine and pharmaceutically acceptable salts or co-crystals thereof, is dosed with an androgen deprivation therapy without resulting in thrombocytopenia as a dose-limiting toxicity.

16. The method according to claim 15, wherein the androgen-deprivation therapy is enzalutamide, apalutamide, darolutamide, or abiraterone.

17. The method according to claim 1, wherein the subject has an activation of the ETS transcription factor family, either through activating mutations and/or translocations, including TMPRSS2-ERG, SLC45A3-ERG, NDRG1-ERG, DUX4-ERG, ELF4-ERG, ELK4-ERG, BZW2-ERG, CIDEC-ERG, DYRK1A-ERG, EWSR1-ERG, FUS-ERG, GMPR-ERG, HERPUD1-ERG, KCNJ6-ERG, ZNRF3-ERG, ETS2-ERG, ETV1-ERG, HNRNPH1-ERG, PAK1-ERG, PRKAB2-ERG, SMG6-ERG, SLC45A3-FL11, TMPRSS2-ETV1, SLC45A3-ETV1, FOXP1-ETV1, EST14-ETV1, HERVk17-ETV1, ERVK-24-ETV1, C150RF21-ETV1, HNRPA2B1-ETV1, ACSL3-ETV1, OR51E2-ETV1, ETV1 S100R, RBM25-ETV1, ACPP-ETV1, BMPR1B-ETV1, CANT1-ETV1, ERO1A-ETV1, CPED1-ETV1, HMGN2P46-ETV1, HNRNPA2B1-ETV1, SMG6-ETV1, FUBP1-ETV1, KLK2-ETV1, MIPOL1-ETV1, SLC30A4-ETV1, EWSR1-ETV1, TMPRSS2-ETV4, KLK2-ETV4, CANT1-ETV4, DDX5-ETV4, UBTF-ETV4, DHX8-ETV4, CCL16-ETV4, EDIL3-ETV4, EWSR1-ETV4, SLC45A3-ETV4, UBTF-ETV4, XPO7-ETV4, TMPRSS2-ETV5, SLC45A3-ETV5, ACTN4-ETV5, EPG5-ETV5, LOC284889-ETV5, RNF213-ETV5, SLC45A3-ELK4.

18. The method according to claim 1, wherein the subject has a spike in PSA either at 4 weeks or 8 weeks of treatment.
